(19) United States
(12) Patent Application Publication
(10) Pub. No.: US 2024/0240193 A1
(43) Pub. Date: Jul. 18, 2024

(54) GENE FOR REGULATING BRANCH NUMBERS OF SOYBEAN AND USE THEREOF

(71) Applicant: Institute of Genetics and Developmental Biology Chinese Academy of Sciences, Beijing (CN)

(72) Inventors: Zhixi TIAN, Beijing (CN); Qianjin LIANG, Beijing (CN); Shulin LIU, Beijing (CN); Min ZHANG; Guoan ZHOU; Yi PAN; Xingtong LU

(21) Appl. No.: 18/000,075

(22) PCT Filed: May 9, 2022
(86) PCT No.: PCT/CN2022/091622

§ 371 Date: Nov. 28, 2022

(51) Int. Cl. C12N 15/82; C07K 14/415; C12N 15/11

(52) U.S. Cl. CPC C12N 15/8205; C07K 14/415; C12N 15/11; C12N 15/8261; C12N 2310/20

(57) ABSTRACT

The present invention provides an isolated gene and promoter for regulating the branch numbers of soybean, so as to regulate the branch numbers of soybean, thereby improving the yield of soybean. The present invention will have a great theoretical and practical value in soybean high-yield breeding and related application research.

### FIELD OF THE INVENTION

The present invention belongs to the field of biotechnology, and particularly relates to a gene for regulating the branch numbers of soybean and use thereof.

### BACKGROUND OF THE INVENTION

Soybean is an important grain and oil crop originated from China. Soybean is rich in protein, is a high-quality source of vegetable protein and an important source of animal feed. It plays an irreplaceable role in all aspects of national economy and life. In recent decades, soybean production in China does not increase rapidly, but consumer demand has grown rapidly. Therefore, it is of great significance to increase soybean yield, so as to ensure food security in China and firmly hold the Chinese people's rice bowl in their own hands.

Plant type of soybean is a key factor affecting its yield. Reasonable spatial configuration has a great influence on photosynthesis efficiency of soybean. The ventilation and light transmission capacity of the crown layer of the soybean can be directly affected by improving plant type-related traits of the soybean such as leaves, branches and internodes, thereby the light interception rate and light energy utilization rate of leaves at the middle and upper portion of soybean will be improved, and the biomass yield of soybean will ultimately be improved (Jie Gao et al., 2017). Branch numbers of soybean is a major trait of plant type. Too many branches will easily consume the nutrients of the plant excessively and affect the podding during the later stage, while too few branches will reduce the flower-pods of the whole plant and reduce the yield. Plant branches are not only determined by genetic factors, but also easily affected by environmental conditions. Therefore, it has important theory and application values for subsequent gene cloning and functional research to understand the development process and production mechanism of branching, and analyzing the molecular regulation mechanism of its formation (Pengtao Gong et al., 2005).

MADS-box transcription factor family genes play an important role in plant growth and development. In Arabidopsis thaliana, they are mainly involved in the process of early inflorescence meristem development and are characteristic genes of floral organ formation (Becker et al., 2003). However, few MADS-box family genes have been identified in soybean (Zheng et al., 2013), or their functions have been identified as mainly related to stem growth habit and flowering (Ping et al., 2014; Liu et al., 2016; Zeng et al., 2018; Zhang et al., 2019), but whether they affect the branch numbers has not been reported.

Therefore, it is of great significance to explore the regulation mechanism of MADS-box family members in soybean on branch development of soybean, and it is of great significance to improve the yield and quality of soybean.

### SUMMARY OF THE INVENTION

In order to further improve the yield and quality of soybeans, the present invention analyzes the genetic regulation sites of complex yield traits such as the branch numbers in soybeans at the whole-genome level by association analysis, and by integrating haplotypes, gene expression profiles, homology gene function annotation and previous QTL information related to soybean branch numbers, using molecular biology and comparative genomics methods, the gene SoyZH13_18 G242900 (Dt2) that regulates soybean branch numbers and yield was cloned. It provides theoretical basis and genetic resources for subsequent molecular assisted breeding and molecular design breeding.

Specifically, the present invention provides the following technical solutions:

In one aspect, the present invention provides an isolated gene regulating branch numbers of legume, preferably genus Glycine, Pisum, Cicer Linn., such as soybean, pea, mung bean, broad bean, black bean, chickpea, wherein the coding region sequence of the gene is shown in SEQ ID NO:3 or SEQ ID NO:4.

In another aspect, the present invention provides a protein that regulates branch numbers of legume, preferably genus Glycine, Pisum, Cicer Linn., such as soybean, pea, mung bean, broad bean, black bean, chickpea, wherein the amino acid sequence of the protein is shown in SEQ ID NO:5 or SEQ ID NO:6.

In another aspect, the present invention provides an isolated promoter of a gene that regulates branch numbers of legume, preferably genus Glycine, Pisum, Cicer Linn., such as soybean, pea, mung bean, broad bean, black bean, chickpea, wherein the sequence of the promoter is shown in SEQ ID NO: 1 or SEQ ID NO: 2.

In another aspect, the present invention provides an expression vector, wherein the expression vector comprises an sgRNA target sequence designed for the above-mentioned gene.

In some embodiments, the expression vector has an antibiotic marker or a chemical resistance marker.

In another aspect, the present invention provides a plant cell into which the above-mentioned expression vector has been introduced.

In another aspect, the present invention provides a gene that regulates branch numbers of legume, preferably genus Glycine, Pisum, Cicer Linn., such as soybean, pea, mung bean, broad bean, black bean, chickpea, in the regulation of branch numbers of soybean and improving yield of the soybean, wherein a nucleotide sequence of the gene is shown in SEQ ID NO:3 and SEQ ID NO:4.

In another aspect, the present invention provides a method for improving yield of legume, preferably genus Glycine, Pisum, Cicer Linn., such as soybean, pea, mung bean, broad bean, black bean, chickpea, or regulating branch numbers of legume, preferably genus Glycine, Pisum, Cicer Linn., such as soybean, pea, mung bean, broad bean, black bean, chickpea, wherein the method comprises a step of inactivating the function of the above-mentioned gene, preferably, function of the gene is inactivated by gene knockout.

In another aspect, the present invention provides a method for improving yield of legume, preferably genus Glycine, Pisum, Cicer Linn., such as soybean, pea, mung bean, broad bean, black bean, chickpea, or regulating branch numbers of legume, preferably genus Glycine, Pisum, Cicer Linn., such as soybean, pea, mung bean, broad bean, black bean, chickpea, wherein the method comprises:
a. designing an sgRNA target sequence for the above-mentioned gene, and introducing the sgRNA target sequence into an expression vector;
b. introducing the expression vector into Agrobacterium tumefaciens; and
c. transfecting legume, preferably genus Glycine, Pisum, Cicer Linn., such as soybean, pea, mung bean, broad bean, black bean, chickpea or a tissue of the legume, preferably genus Glycine, Pisum, Cicer Linn., such as soybean, pea, mung bean, broad bean, black bean, chickpea with the Agrobacterium tumefaciens.

In some embodiments, the sgRNA target sequence is shown in SEQ ID NO:9 or SEQ ID NO:10.

In some embodiments, the expression vector is selected from PMDC123 and PTF101.

In some embodiments, the Agrobacterium is selected from a group consisting of EHA105, EHA101 and GV3101.

In some embodiments, the transfection method is selected from a cotyledonary node transformation method and an embryo tip explant transformation method.

### Definitions

BLUP data: the Best Linear Unbiased Prediction (referred to as BLUP) belongs to the application of mixed linear models. Mainly through the principle of least squares, the obtained estimated value is the smallest unbiased estimate of the error variance. This model can combine multiple records, multiple traits, and factors under multiple environment to perform comprehensive estimation, minimize the interference of environmental and human errors on phenotype data, so as to reflect the relationship between phenotype and genotype better.

QQ plot: Quantile-Quantile plot is a probability plot method for comparing two probability distributions by comparing the quantiles of these two probability distributions, mainly reflecting whether the actual sample points conform to a probability distribution.

Promoter: it usually refers to a DNA sequence upstream of the transcription initiation site that contains a region capable of binding RNA polymerase and is responsible for regulating the transcription of a gene. The promoter sequence is the DNA molecule shown in FIG. 2, i.e. a sequence located 3500-bp upstream of the Dt2 start codon, including two types of sequences. The promoter sequences of the present invention are shown in SEQ ID NO: 1 and SEQ ID NO: 2, which are functional in the transient transfection promoter activity experiment, showing that SEQ ID NO: 2 has a stronger promoter activity. The transient transfection promoter activity experiment uses the dual luciferase reporter system, and links SEQ ID NO: 1 and SEQ ID NO: 2 sequences to a reporter vector, respectively and transform the reporter vector into Agrobacterium tumefaciens GV3101, after infecting tobacco leaves, luciferase detection instrument is used to detect the fluorescein intensity of different reporter vectors, so as to compare the promoter activity of different sequences to reporter genes.

CDS (Coding sequence): coding region, is a DNA sequence capable of encoding a protein, containing a start codon and a stop codon. The CDS sequence of the present invention contains a MADS-box domain and a K-box domain.

Negative regulation: a regulation that can inhibit or attenuate the level of gene transcription. Usually, the repressor protein binds to the operator gene so that RNA polymerase cannot bind to the promoter, thus transcription is inhibited. The present invention confirms that the gene obtained by the present invention is a negative regulation gene through gene overexpression, and the expression of the gene leads to decrease in the branch numbers of a plant, decrease in plant height, and significant decrease in yield per plant and plot yield.

sgRNA target sequence: sgRNA is a small guide RNA (guide RNA, gRNA), which guides the insertion or deletion of uridine residues into kinetoplasts during the process of RNA editing. It is a small non-coding RNA, and can be paired with pre-mRNA. Guide RNA-edited RNA molecules, about 60 to 80 nucleotides in length, are transcribed from a separate gene, and have a 3′ oligo U tail, a sequence that is exactly complementary to the edited mRNA in the middle, and an anchor sequence at the 5′ end that is complementary to the unedited mRNA sequence.

CRISPR-Cas9: CRISPR-Cas9 technology includes two important components, one is the Cas9 protein that performs the function of DNA double-strand cleaving, and the other is the gRNA (guide RNA) that performs the function of guiding. CRISPR-Cas9 technology uses a segment of gRNA complementary to the target sequence to guide Cas9 nuclease to recognize and cleave the specific target DNA, resulting in breaking of double-stranded or single-stranded DNA. The cell then uses its own DNA repair mechanism to repair the broken DNA, resulting in base mismatch or deletion, so as to achieve the function of directional editing of the target gene. With the rapid development of molecular biology in recent years, it has been widely used in many fields. It can not only be used for the study of expression regulation and gene function, the construction of cell animal models, the screening of oncogenes and drug targets, but also has great development prospects in gene therapy, providing new treatment methods for a variety of diseases.

Homologous sequence: refers to the homology between different species within orthologous in particular, such as protein homology, DNA sequence homology.

### Beneficial Effects

The nucleotides of the gene related to branch numbers of soybean and proteins encoded by the gene provided by the present invention are firstly discovered by the applicant, and the phenotypic analysis of the transgenic plants shows that the soybean branch number-related proteins of the present invention can negatively regulate the branch numbers of transgenic plants of soybean.

The present invention will have great theoretical and application value in high-yield breeding and related application research of soybean.

### BRIEF DESCRIPTION OF THE DRAWINGS

FIG. 1 shows the cloning of the SoyZH13_18 G242900 (W82 version gene number: Glyma. 18G273600, also known as Dt2 or Dt2^{HapI-2}) gene. Among them, A: whole-genome association analysis of branch number trait BLUP (breeding value) data of soybean; B: QQ (normal distribution) dot plot of branch number trait; C: association analysis display of branch numbers on chromosome 18; D: schematic diagram of the size distribution of all genes in the candidate interval (where the white box is the location of the candidate gene) and linkage analysis of all SNPs in the candidate interval; E: analysis of the expression pattern of genes in the linkage association region; F: schematic diagram of the structure of Dt2 gene; G: three main haplotype structures of Dt2 (among them, HapI-1 is a multi-branched haplotype; HapI-2 is an intermediate-branched haplotype, and HapII is a less-branched haplotype). a, b represent the significance distribution of Duncan's multiple range test. H: Correlation between branch numbers and Dt2 expression level in 14 Dt2 haplotype I materials (grey circles) and 6 haplotype II materials (black circles). I: Analysis of promoter activity between Dt2 haplotype I and haplotype II. where ** represents P<0.01.

FIG. 2 shows a schematic diagram of the nucleotide sequences of two promoters located at 3500 bp upstream of the start codon of the SoyZH13_18 G242900 (W82 version gene number: Glyma. 18G273600, also known as Dt2) gene. Among them, the bases with red darkened background are the position of the mutated base of Dt2 promoter.

FIG. 3 shows a schematic diagram of the CDS nucleotide sequences of two haplotype of the SoyZH13_18 G242900 (W82 version gene number: Glyma.18G273600, also known as Dt2) gene. Among them, the bases with red darkened background are the position of the mutation bases in the Dt2 coding region.

FIG. 4 shows a schematic diagram of the amino acid sequences of the two haplotype proteins of the SoyZH13_18 G242900 (W82 version gene number: Glyma. 18G273600, also known as Dt2) gene. Among them, the bases with red darkened background are the position of mutated base of Dt2 amino acid sequence.

FIG. 5 shows a schematic diagram of the CRISPR-Cas9 target designed specific for the SoyZH13_18 G242900 (W82 version gene number: Glyma. 18G273600, also known as Dt2) gene. Among them, A: Dt2-specific target design, sequence in capital letter represents the target sequence; B: structural schematic diagram of the partial vector of the target sequence sgRNA in the CRISPR/Cas9 system. C-D: distinct edited individual plant events of Dt2. Among them, C is Dt2 knockout line 1, with one base missing; D is Dt2 knockout line 2, with 19 bases missing.

FIG. 6 shows the statistical comparison of the phenotype of the knockout mutants of the SoyZH13_18 G242900 (W82 version gene number: Glyma. 18G273600, also known as Dt2 or Dt2^{HapI-2}) gene and the wild-type (Dongnong 50). Among them, A-B: comparison of field phenotype of DN50 wild-type and transgenic plants of Dt2-knockout mutants, the scale bar is 10 cm;

FIG. 7 shows a schematic map of the SoyZH13_18 G242900 (W82 version gene number: Glyma. 18G273600, also known as Dt2 or Dt2^{HapI-2}) gene transformation vector 35S::Dt2^{HapI-2}.

FIG. 8 shows the statistical comparison of the phenotype of the wild type (Dongnong 50) and the transgenic plant which overexpresses Dt2 by transfection with the 35S::Dt2^{HapI-2} plasmid. Among them, A: field phenotype comparison of DN50 wild type and Dt2 overexpression transgenic plants, scale bar is 10 cm; B: comparison of branching ability of DN50 wild type and Dt2 overexpression transgenic plants, the scale bar is 10 cm; C: Dt2 expression level analysis of different Dt2 overexpressing family lines.

FIG. 9 shows the statistics analysis of branch numbers, plant height, flowering time, 100-seed weight, grain weight per plant and plot yield between Dt2 overexpression lines, Dt2 knockout lines and wild-type Dongnong 50 materials, including the sample size, data range, mean±standard deviation, and the significance of the ratio of Dt2 overexpression lines, and Dt2 knockout lines to wild-type Dongnong 50, the significance test was performed by two-tailed T test.

FIG. 10 shows statistical comparison of plot yield between Dt2 knockout lines and wild type (Dongnong 50) at different planting densities.

### EXAMPLES

In order to make the objectives, technical solutions and advantages of the present invention more clearly understood, the present invention will be further described in detail below in conjunction with examples and with reference to the accompanying drawings.

The following examples are intended to facilitate a better understanding of the present invention, but are not intended to limit the present invention. The quantitative tests in the following examples are carried out in triplicate, and the results are averaged.

In the following examples, the transformation receptor is Dongnong 50 (DN50), and DN50 is an approved variety of Heilongjiang Province (Heishendou 2007022), which can be purchased from the market. The pTF101.1 vector and Agrobacterium strains EHA101 and EHA105 were purchased from the China Plasmid Vector Strain Cell Protein Antibody Gene Collection Center (Biovector Science Lab, Inc).

Consumables such as enzyme digestion and recovery kit were purchased from New England Biolabs and Tiangen Biochemical Technology (Beijing) Co., Ltd.

### Example 1. Analysis of Variation in Dt2 Gene, Encoded Protein Thereof, and the Promoter

The inventors have performed phenotypic identification on the branch numbers for two years, performed BLUP processing on the results and combined whole-genome association analysis, and found a stable signal closely related to the branch numbers on chromosome 18. Combined with transcriptome data, and based on a large number of sequence analysis and functional verification, a protein encoding soybean branch number gene was found, which was named as Dt2 protein (referred to as Dt2), and the gene encoding Dt2 protein was named as Dt2 (or Dt2^{HapI-2}) gene.

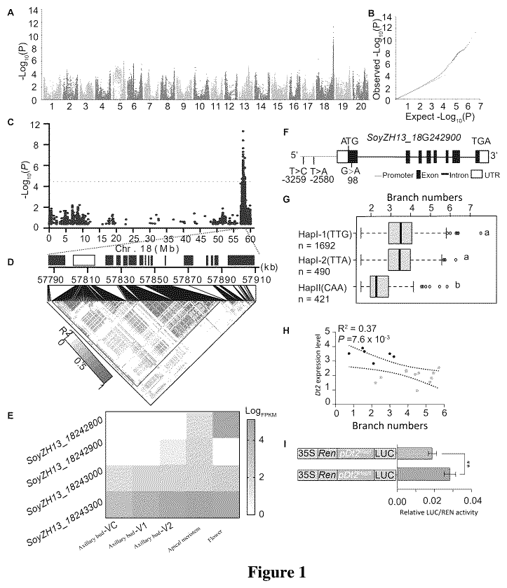

Further analysis of the gene structure of this gene revealed that there were two major variations with significant correlation at 3259 bp and 2580 bp upstream of ATG promoter region, and a non-synonymous mutation that could change the encoded amino acid was found at 98 bp in the coding region. (FIG. 1F). By haplotype analysis on the three variations, it was found that (FIG. 1G) the three variants can be divided into three types of haplotypes: haplotype HapI-1 (Dt2^{HapI-1}), haplotype HapI-2 (Dt2^{HapI-2}), haplotype HapII (Dt2^{HapII}). Considering that only the haplotype HapII has significant differences, it shows that the variation of the promoter region can significantly affect the change of branch number. Therefore, we randomly selected a total of 16 materials of haplotype HapI and haplotype HapII in proportion to analyze the correlation between gene expression level and branch numbers, and found that gene expression level was significantly negatively correlated with branch numbers (FIG. 1H). The promoters of haplotype I and haplotype II were respectively linked to the reporter vector, and binary luciferase transient transfection expression experiment was performed in protoplasts of Arabidopsis thaliana, and it was found that the promoter activity of haplotype II was significantly stronger than that of haplotype I, indicating that the variation in promoter region can affect the expression level of Dt2 gene (FIG. 1 I).

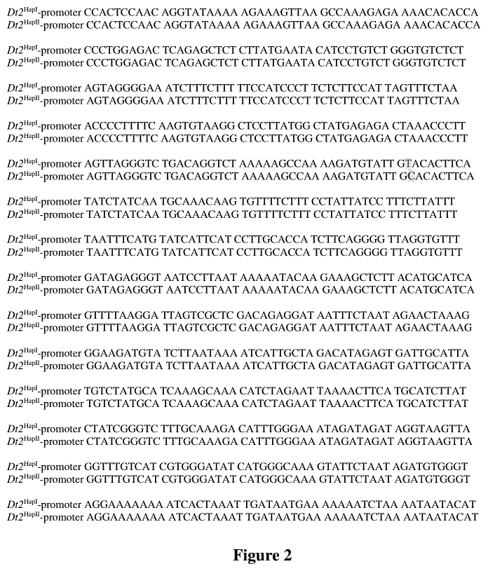

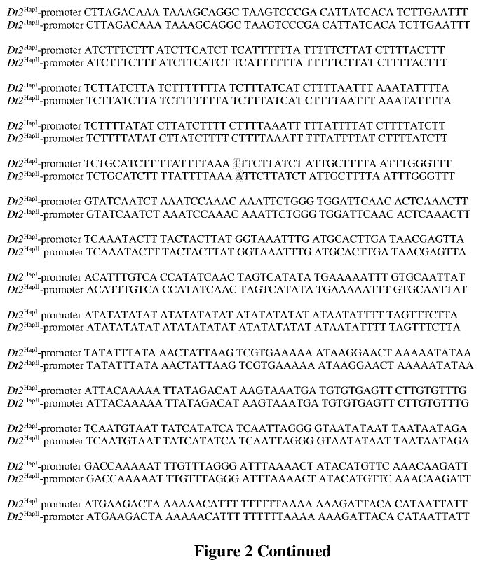

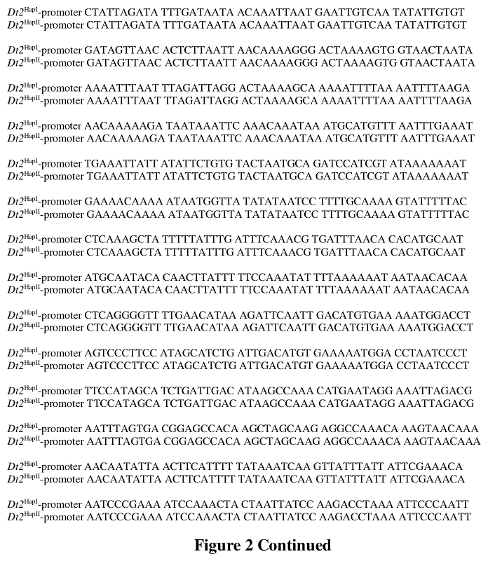

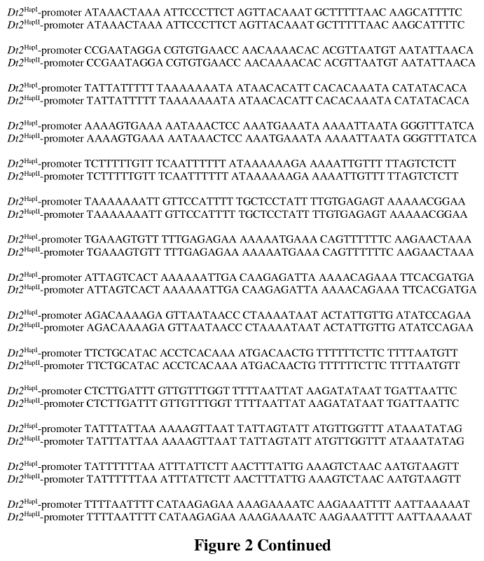

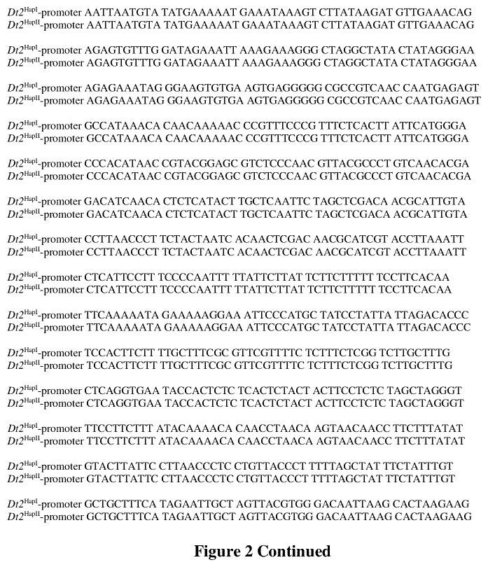

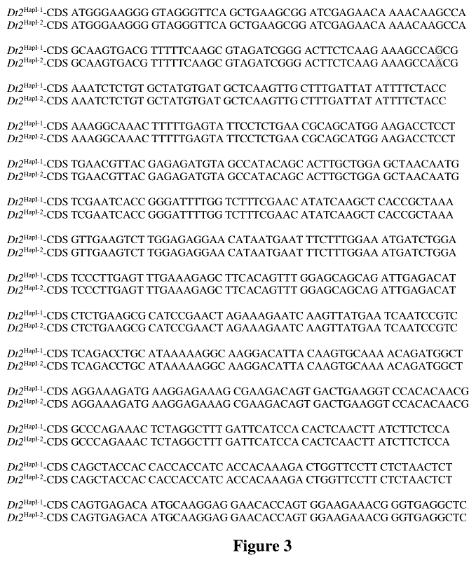

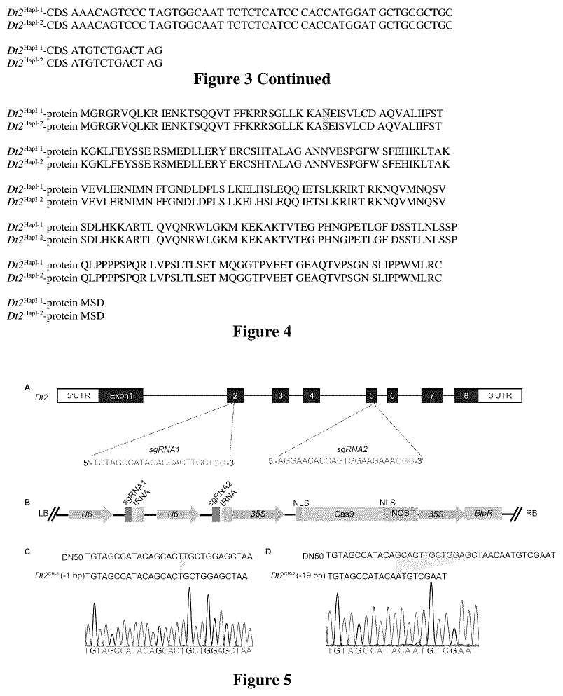

The present invention further provides the nucleotide polymorphism of the SNP site of the Dt2 gene in the soybean genome, such as the sequence difference sites shown in FIGS. 2 and 3, which can be used to detect the background of soybean varieties.

### Example 2. Functional Verification of Dt2 Protein

### I. Construction of Recombinant Plasmids

RNA was extracted from wild-type Dongnong 50 (DN50) and reverse-transcribed into cDNA. The Dt2^{HapI-2} gene cDNA was amplified by PCR, and then digested and ligated into the PTF101.1 vector to obtain the recombinant plasmid 35S::Dt2^{HapI-2}. The specific operations were as follows:
1. Separate the leaves of soybean variety DN50 from the plant, extract RNA, and obtain leaf RNA of soybean variety DN50.
2. The total RNA obtained in step 1 was reverse-transcribed into cDNA using a reverse transcription kit (TransGen Biotech). The obtained cDNA as used as the template, PCR amplification was carried out with the primer pair composed of F1 and R1, and the PCR amplification product was obtained (because the genotype of wild-type Dongnong 50 is Dt2^{HapI-2}, we chose self-genotype Dt2^{HapI-2} to construct the vector after considering the influence of the self genotype).

F1:
(SEQ ID NO: 7)
5′-ATGGGAAGGGGTAGGGTTC-3′;
R1:
(SEQ ID NO: 8)
5′-CTAGTCAGACATGCAGCGCA-3′.

3. The PTF101.1 vector (purchased from the China Plasmid Vector Strain Cell Protein Antibody Gene Collection Center) was double digested with restriction endonucleases XbaI and SacI, and the vector backbone of about 9138 bp was recovered.
4. The PCR product of step 2 and the vector backbone of step 3 were linked to obtain a recombinant plasmid 35S::Dt2^{HapI-2} (see FIG. 7 for its map).

### II. Obtaining of Dt2^{HapI-2} Overexpressing Transgenic Plants

1. The recombinant plasmid 35S::Dt2^{HapI-2} was introduced into Agrobacterium tumefaciens strain EHA101 (purchased from China Plasmid Vector Strain Cell Protein Antibody Gene Collection Center) to obtain recombinant Agrobacterium tumefaciens.
2. The recombinant Agrobacterium tumefaciens obtained in step 1 was transformed into the recipient plant DN50 using the cotyledonary node transformation method (Margieet et al., 2004), and the T₀ generation seeds were harvested. The specific steps were as follows:

### (1) Seed Sterilization and Germination

DN50 (TL) soybean seeds with round kernels, smooth surface and no disease spots were selected and placed in a 120 mm petri dish. The petri dish was putted into a desiccator with a beaker, 100 ml of sodium hypochlorite solution and 4 ml of concentrated hydrochloric acid were added into the beaker, the lid of the desiccator was immediately closed, and the soybean seeds were sterilized with chlorine gas for 18 h. The lid was opened in the clean bench to blow off the residual chlorine gas.

### (2) Seed Germination

The sterilized soybean seeds were placed with the hilum facing down evenly in the germination medium GM, 30 to 35 seeds per dish. Then the dish was wrapped with a fresh-keeping bag, ventilation opening was cut in the fresh-keeping bag, and the dish was put into a dark incubator. The germination condition was 22° C. for more than 16 hours.

### (3) Explant Preparation

The germinated seeds were taken, the seeds were longitudinally cut into two symmetrical parts along the hypocotyl, a pair of euphylla at the cotyledonary node was gently scraped under a microscope, and finally, the cotyledonary nodes pricked with a scalpel for several times were the explants used for transformation.

(4) Preparation of Agrobacterium tumefaciens Infection Solution

The recombinant Agrobacterium tumefaciens 35S::Dt2^{HapI-2}-EHA101 stored in glycerol and frozen at −80° C. was taken out and thawed on ice, then a small amount of bacteria solution was dipped with a sterilized pipette tip in an ultra-clean bench and streak cultured in YEP solid medium containing Kan (Kanamycin) and Spe (spectinomycin), activated and cultured at 28° C. for 2 days, and then spread on new YEP solid medium containing Kan and Spe with a spreader, and cultured overnight, and finally the Agrobacterium tumefaciens cultured overnight was resuspended with infection solution to an OD₆₀₀ value of 0.6. In this step, the components of YEP solid medium were: 1 g/L yeast extract, 5 g/L peptone, 0.5 g/L MgSO₄·7H₂O, 5 g/L beef extract, 5 g/L sugar, 15 g/L agar, pH 5.7; the composition of the infection solution was: 0.31 g/L B5 (basal salts of B5 medium, Phytotech, Cat. No. HYY0768022E), 30 g/L glucose, 3.9 g/L MES (Kanamycin sulfate), pH 5.5, 1% c (w/v) Vitamin B5, 0.25%% (w/v) Gibberellin GA3, 1.67%% (w/v) Hexabenzylpurine 6-BA, 1.8%% (w/v) Dithiothreitol DTT, 0.8% c (w/v) acetosyringone AS.

(5) Agrobacterium tumefaciens Infection and Co-Culture of Explants

The previously prepared explants for transformation were put into the resuspended Agrobacterium tumefaciens bacteria solution, placed in a 22° ° C. dark incubator to infect overnight, and then the excess bacteria solution on the surface was absorbed with sterile filter paper. Cotyledonary nodes were plated on solid co-culture medium spread with sterile filter paper thereon and infected in the dark at 22° C. for 5 days. The solid medium was supplemented with 9 g/L agar in addition to the liquid medium.

### (6) Obtaining Transgenic Seedlings

After 5 days of co-cultivation, the cotyledonary nodes were inserted obliquely into bud induction medium I, at 25° C., 16 h light and 8 h dark cycle, and the light intensity was 5000 to 6000 Lux. After 7 days of recovery, the buds were transferred to bud induction medium II containing 8 mg/ml of PPT (glufosinate-ammonium) and continued to culture for 14 to 20 days. The cluster buds were excised from the hypocotyls and transferred to bud elongation medium containing 4 mg/ml of PPT, at 25° ° C., 16 h light and 8 h dark cycle, light intensity was 5000 to 6000 Lux, and subcultured every 10 days until the buds elongate to about 5 cm. The sprouts that have grown to about 5 cm were cut off, and inserted straightly into the rooting medium, at 25° C., 16 h light and 8 h dark cycle, and the light intensity was 5000 to 6000 Lux, until the roots elongated to 3 to 4 cm, ready for transplanting.

In this step, the bud induction medium I was composed of 3.1 g/L B5 salt (basal salt of B5 medium), 1% (w/v) B5 vitamin, 30 g/L sucrose, 0.6 g/L kanamycin sulfate MES, 1.6 mg/L hexabenzylpurine 6-BA, 50 mg/L cephalosporin, 150 mg/L Tim, 4 g/L glufosinate-ammonium, 0.2% (w/v) plant gel, pH 5.7; bud induction medium II was composed of 3.1 g/L B5 salts, 1% c (w/v) B5 vitamins, 30 g/L sucrose, 0.6 g/L MES, 1.6 mg/L 6-BA, 50 mg/L cephalosporin, 150 mg/L Tim, 8 g/L glufosinate-ammonium, 0.2% (w/v) plant gel, pH 5.7; bud elongation medium was composed of 4.33 g/L MS salts (MS medium Basal Salt, Phytotech, Cat. No.: HHY0524225A), 1% c (w/v) Vitamin B5, 30 g/L Sucrose, 0.6 g/L MES, 0.5 mg/L gibberellin GA3, 1 mg/L Zeatin ZR, 50 mg/L L-Glu Glufosinate-ammonium, 50 mg/L Aspartate Asp, 0.1 mg/L Auxin IAA, 50 mg/L Cephalosporin Cef, 100 mg/L Tim, 4 g/L Glufosinate-ammonium, 0.2% (w/v) plant gel, pH 5.8; rooting medium was composed of 2.165 g/L MS salts (MS medium basal salts), 1% c (w/v) B5 vitamins, 20 g/L sucrose, 0.6 g/L MES, 50 mg/L L-Glu, 50 mg/L Aspartate Asp, 1.5 mg/L Indolebutyric Acid IBA, 25 mg/L Tim, 0.2% (w/v) plant gel, pH 5.8.

### (7) Seedling Transplanting and Screening

The sealing film was removed from the tissue cultured seedlings to be transplanted, add a small amount of sterile water was added, at 25° C., 16 h light and 8 h dark cycle, and light intensity was 5000 to 6000 Lux. After culturing for two days, the seedlings were transplanted, and the same amount of vermiculite and turfy soil was mixed, and put into a tray added with water, then the tissue cultured seedlings were pulled out from the rooting medium, the residual medium at the roots was rinsed off, and the seedlings were moved into nutrient soil that was fully saturated with water. Soybean leaves were smeared with 0.1% Basta herbicide, and if there was no etiolation reaction in the plants after 3 days, positive transgenic plants were obtained.

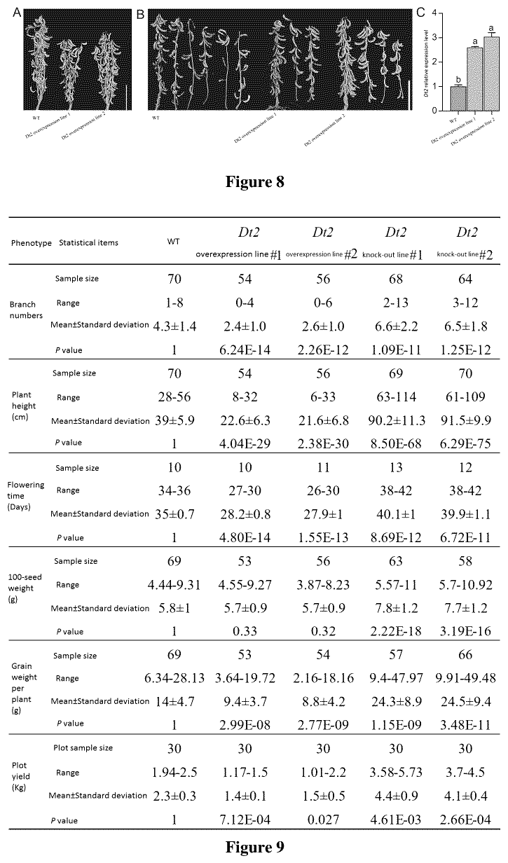

Subsequent T1 generation and subsequent generations of transgenic lines were sprayed with 0.1% Basta herbicide (Coolab, CB2471-100 mL) for screening, and if all of the transgenic lines were resistant to Basta, then the transgenic plants successfully transformed with the recombinant plasmid 35S::Dt2^{HapI-2} were obtained (that was, the plants overexpressing Dt2). Among them, #1 and #2 in FIG. 8 refer to the names of the transgenic lines, that was, a total of two transgenic lines were obtained. For the Dt2 overexpression line, it refers to the transgenic plant obtained by transfection with the 35S::Dt2^{HapI-2} plasmid (FIG. 7, FIG. 8), and the wild type refers to the DN50 plant; and whether the overexpression was successful was determined by extracting RNA and detecting the expression level, it was found that the expression levels of the two overexpressing lines of Dt2 were significantly increased by real-time quantitative PCR (FIG. 8C), indicating that the expression levels of Dt2 in the transgenic lines were increased.

### III. Obtaining of Dt2 CRISPR-Cas9 Knockout Mutant Plants

1. The Dt2 target sequence was constructed into the binary expression vector PMDC123 plasmid (purchased from the China Plasmid Vector Strain Cell Protein Antibody Gene Collection Center (Biovector NTCC)). Specific steps were as follows:

### (1) The Dt2 Target Sequence was Linked to the Intermediate Vector U6

The target sequence primers sgRNA1 (there was no difference between the two sgRNAs in Dt2^{HapI-1} and Dt2^{HapI-2} materials) (5′-tgtagccatacagcacttgc-3′, SEQ ID NO: 9), sgRNA2 (5′-aggaacaccagtggaagaaa-3′, SEQ ID NO: 10) were dissolved in sterilized water to obtain a 10 M stock solution, and 10 μL of each primer was added into 80 μL of 0.5×TE (pH 8.0), the final concentration of each primer was 1 μM. The reactant was then heated at 98° C. for 3 min in a PCR instrument, naturally cooled to room temperature and placed at room temperature for more than 2 h. The U6 vector was digested with BsaI, and the sgRNA was ligated with U6 (purchased from the China Plasmid Vector Strain Cell Protein Antibody Gene Collection Center) using T4 ligase, transformed into E. coli, and spread onto Amp-resistant LB solid medium, cultured overnight at 37° ° C., colonies that are sequenced correct were picked to obtain sgRNA1-U6 and sgRNA2-U6 containing the target sequence.

(2) Dual-Target sgRNA-U6 Tandem

The sgRNA2-U6 was digested with SpeI and Nhe, and the product of about 750 bp was recovered as an insert fragment; at the same time, the sgRNA1-U6 vector was digested with SpeI, and the vector of about 13,658 bp was recovered as the backbone vector. The backbone vector was linked to the insert fragment (sgRNA2-U6 double-enzyme digestion product) by T4 ligase, transformed into E. coli, and the E. coli was plated on Amp-resistant LB solid medium, cultured overnight at 37° ° C., and picked out the colonies which were sequenced correct, that was, sgRNA-U6 containing dual targets (sgRNA1+sgRNA2, the purpose of constructing dual targets was to increase the probability of gene editing and improve the efficiency of gene editing) was obtained.

(3) Dual-Target sgRNA-U6 was Seamlessly Cloned into the Final Vector PMDC123 Plasmid

The final vector PMDC123 plasmid was digested with HindIII and PstI, and the fragment was recovered as the backbone vector. At the same time, the sgRNA-U6 containing dual targets was amplified with primers containing homologous arms. Seamless linking was performed with Homologous Recombination Kit of TransGen Biotech (Cat. No. CU101-01), the product was transformed into Escherichia coli, and the Escherichia coli was plated on Kan-resistant LB solid medium, cultured overnight at 37° ° C., colonies that were sequenced correct were picked out, and PMDC123 plasmid having dual targets and pro: U6-sgRNA-Cas9 driven by U6 promoters was obtained (FIG. 5B). Among them, the sequences of the amplification primers carrying the homologous arm (the homologous arm was partial sequence on the final vector PMDC123, the amplification primer sequence was partial sequence on the dual-target sgRNA-U6, and the underlined portion was the homologous arm) were:

PMDC123-HindIII-gRNA-F (SEQ ID NO: 11):
TGTAAAACGACGGCCAGTGCCAAGCTTACGACTCACTATAGGGCG
AATTG
PMDC123-pstI-gRNA-R (SEQ ID NO: 12):
GTGCTCCACCATGTTGACCTGCAGAACAAAAGCTGGAGCTCACTA
GT

2. The correctly constructed plasmid was introduced into Agrobacterium tumefaciens strain EHA105 (purchased from China Plasmid Vector Strain Cell Protein Antibody Gene Collection Center) by electroporation method to obtain recombinant Agrobacterium tumefaciens.

Specific steps were as follows:
(1) About 150 ng of plasmid was added into the competent Agrobacterium tumefaciens strain EHA105 and placed into ice for 30 minutes;
(2) the electrode cup was placed in the ultra-clean bench and blow until there was no ethanol, and then put into ice to cool for later use;
(3) the competent EHA105 strain mixed with the plasmid was added to the electrode cup, and shocked with an electric shock meter; (4) about 700 ul of antibiotic-free LB medium was added into the competent EHA105 after electric shock, the EHA105 was placed on a shaker at 30° ° C. to activate at 220 rpm for 1 hour, and then plated on Kan (kanamycin) and Rif (rifampicin) resistant medium, cultured for 2-3 days, correct colony was identified by colony PCR, and reactivated to obtain the recombinant Agrobacterium tumefaciens to be transformed.
2. The recombinant Agrobacterium tumefaciens obtained in step 1 was transformed by the cotyledonary node transformation method (for the specific operation method, please refer to the overexpression construction transformation) into the recipient plants DN50, and the seeds of the T₀ generation were harvested. The positive homozygous rate by editing was identified, so as to obtain stably expressed mutants of two different editing types. The positive homozygous rates of Dt2 knockout lines 1 and 2 were 16.7% and 8%, respectively (FIG. 5). In Dt2 knockout line 1, the first target had one base deleted, causing the protein sequence to be premature terminated at the amino acid position 107; in Dt2 knockout line 2, the first target had 19 bases deleted, causing the protein sequence to be premature terminated at the amino acid position 77. Both of the two mutants resulted in lost of protein function.

### Example 3. Statistics of Transgenic Phenotype of Dt2

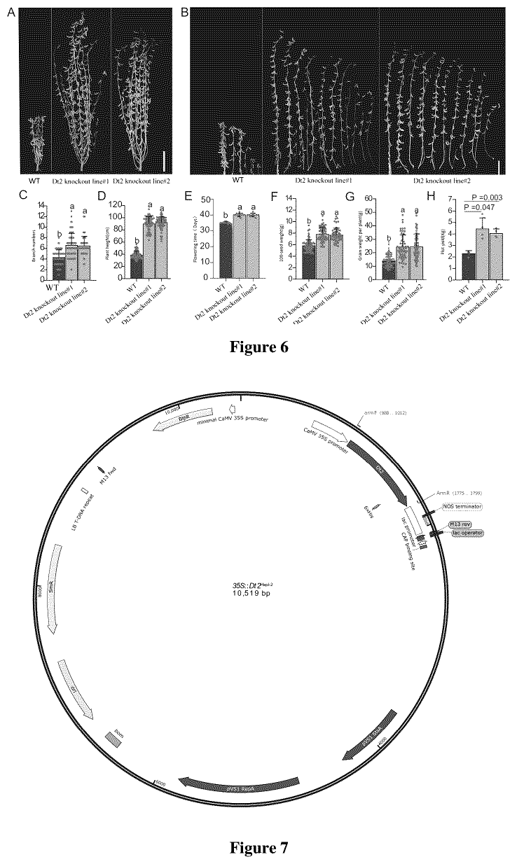

This example confirmed that the branch numbers of the knockout transgenic plants obtained after knocking out Dt2 in the DN50 background increased, and the average percentage of branch numbers increased by 53.7% and 50.3%, respectively, accompanied by increased plant height (plant height increased 131.4% and 134.6%, respectively), delayed flowering, increased 100-seed weight (100-seed weight increased 33.9% and 31.8%, respectively), and significantly increased yield per plant (yield per plant increased 73.1% and 74.7%, respectively) and plot yield (the percentage of plot yield increase was 93.2% and 76.2%, respectively) (FIG. 6, FIG. 9).

This example confirmed that after DN50 was successfully transformed with the recombinant plasmid 35S::Dt2^{HapI-2}, the obtained overexpressed transgenic plants had fewer branches, decreased plant height, earlier flowering time, while no change in 100-seed weight. Yield per plant and plot yield were significantly reduced (FIG. 8, FIG. 9).

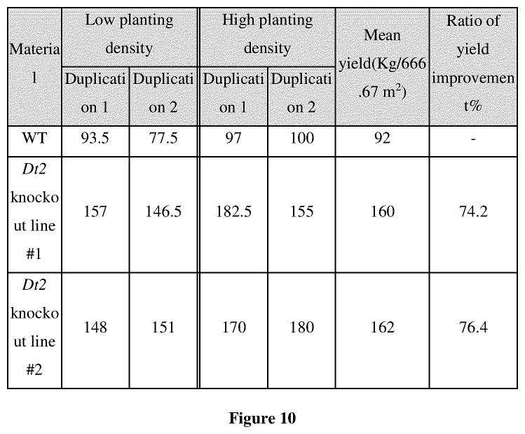

In addition, in order to verify whether Dt2 knockout transgenic lines have an effect on yield, we planted Dt2 gene knockout lines and wild type in Changping Farm, Beijing at low planting density (8800 plants/mu) and high planting density (13340 plants/mu), respectively (FIG. 10). Through the identification of the plot yield in the later stage, it was found based on yield per mu that the Dt2 knockout line can significantly increase the yield, and the average yield was more than 70% higher than that of the control, which can significantly affect the yield. Among them, the yield of Dt2 knockout line #1 was increased by 74.2% compared to the wild type, and the yield of Dt2 knockout line #2 was increased by 76.4% compared to the wild type.

The above results indicated that Dt2 gene was a key gene regulating the branch numbers of soybean. Knockout of Dt2 gene in soybean plants can facilitate the increase of the 100-seed weight of soybean, thereby improving soybean yield.

The examples described above further describe the purpose, technical solutions and beneficial effects of the present invention in further detail. It should be understood that the above examples are only specific examples of the present invention, and are not intended to limit the present invention. Any modifications, equivalent replacements, improvements, etc. made within the spirit and principle of the present invention should be within the scope of the present invention.

Sequences

SEQ ID NO: 1 Sequence of Dt2^{HapI} promoter
CCACTCCAAC AGGTATAAAA AGAAAGTTAA GCCAAAGAGA
AAACACACCA CCCTGGAGAC TCAGAGCTCT CTTATGAATA
CATCCTGTCT GGGTGTCTCT AGTAGGGGAA ATCTTTCTTT
TTCCATCCCT TCTCTTCCAT TAGTTTCTAA ACCCCTTTTC
AAGTGTAAGG CTCCTTATGG CTATGAGAGA CTAAACCCTT
AGTTAGGGTC TGACAGGTCT AAAAAGCCAA AAGATGTATT
GTACACTTCA TATCTATCAA TGCAAACAAG TGTTTTCTTT
CCTATTATCC TTTCTTATTT TAATTTCATG TATCATTCAT
CCTTGCACCA TCTTCAGGGG TTAGGTGTTT GATAGAGGGT
AATCCTTAAT AAAAATACAA GAAAGCTCTT ACATGCATCA
GTTTTAAGGA TTAGTCGCTC GACAGAGGAT AATTTCTAAT
AGAACTAAAG GGAAGATGTA TCTTAATAAA ATCATTGCTA
GACATAGAGT GATTGCATTA TGTCTATGCA TCAAAGCAAA
CATCTAGAAT TAAAACTTCA TGCATCTTAT CTATCGGGTC
TTTGCAAAGA CATTTGGGAA ATAGATAGAT AGGTAAGTTA
GGTTTGTCAT CGTGGGATAT CATGGGCAAA GTATTCTAAT
AGATGTGGGT AGGAAAAAAA ATCACTAAAT TGATAATGAA
AAAAATCTAA AATAATACAT CTTAGACAAA TAAAGCAGGC
TAAGTCCCGA CATTATCACA TCTTGAATTT ATCTTTCTTT
ATCTTCATCT TCATTTTTTA TTTTTCTTAT CTTTTACTTT
TCTTATCTTA TCTTTTTTTA TCTTTATCAT CTTTTAATTT
AAATATTTTA TCTTTTATAT CTTATCTTTT CTTTTAAATT
TTTATTTTAT CTTTTATCTT TCTGCATCTT TTATTTTAAA
TTTCTTATCT ATTGCTTTTA ATTTGGGTTT GTATCAATCT
AAATCCAAAC AAATTCTGGG TGGATTCAAC ACTCAAACTT
TCAAATACTT TACTACTTAT GGTAAATTTG ATGCACTTGA
TAACGAGTTA ACATTTGTCA CCATATCAAC TAGTCATATA
TGAAAAATTT GTGCAATTAT ATATATATAT ATATATATAT
ATATATATAT ATAATATTTT TAGTTTCTTA TATATTTATA
AACTATTAAG TCGTGAAAAA ATAAGGAACT AAAAATATAA
ATTACAAAAA TTATAGACAT AAGTAAATGA TGTGTGAGTT
CTTGTGTTTG TCAATGTAAT TATCATATCA TCAATTAGGG
GTAATATAAT TAATAATAGA GACCAAAAAT TTGTTTAGGG
ATTTAAAACT ATACATGTTC AAACAAGATT ATGAAGACTA
AAAAACATTT TTTTTTAAAA AAAGATTACA CATAATTATT
CTATTAGATA TTTGATAATA ACAAATTAAT GAATTGTCAA
TATATTGTGT GATAGTTAAC ACTCTTAATT AACAAAAGGG
ACTAAAAGTG GTAACTAATA AAAATTTAAT TTAGATTAGG
ACTAAAAGCA AAAATTTTAA AATTTTAAGA AACAAAAAGA
TAATAAATTC AAACAAATAA ATGCATGTTT AATTTGAAAT
TGAAATTATT ATATTCTGTG TACTAATGCA GATCCATCGT
ATAAAAAAAT GAAAACAAAA ATAATGGTTA TATATAATCC
TTTTGCAAAA GTATTTTTAC CTCAAAGCTA TTTTTATTTG
ATTTCAAACG TGATTTAACA CACATGCAAT ATGCAATACA
CAACTTATTT TTCCAAATAT TTTAAAAAAT AATAACACAA
CTCAGGGGTT TTGAACATAA AGATTCAATT GACATGTGAA
AAATGGACCT AGTCCCTTCC ATAGCATCTG ATTGACATGT
GAAAAATGGA CCTAATCCCT TTCCATAGCA TCTGATTGAC
ATAAGCCAAA CATGAATAGG AAATTAGACG AATTTAGTGA
CGGAGCCACA AGCTAGCAAG AGGCCAAACA AAGTAACAAA
AACAATATTA ACTTCATTTT TATAAATCAA GTTATTTATT
ATTCGAAACA AATCCCGAAA ATCCAAACTA CTAATTATCC
AAGACCTAAA ATTCCCAATT ATAAACTAAA ATTCCCTTCT
AGTTACAAAT GCTTTTTAAC AAGCATTTTC CCGAATAGGA
CGTGTGAACC AACAAAACAC ACGTTAATGT AATATTAACA
TATTATTTTT TAAAAAAATA ATAACACATT CACACAAATA
CATATACACA AAAAGTGAAA AATAAACTCC AAATGAAATA
AAAATTAATA GGGTTTATCA TCTTTTTGTT TCAATTTTTT
ATAAAAAAGA AAAATTGTTT TTAGTCTCTT TAAAAAAATT
GTTCCATTTT TGCTCCTATT TTGTGAGAGT AAAAACGGAA
TGAAAGTGTT TTTGAGAGAA AAAAATGAAA CAGTTTTTTC
AAGAACTAAA ATTAGTCACT AAAAAATTGA CAAGAGATTA
AAAACAGAAA TTCACGATGA AGACAAAAGA GTTAATAACC
CTAAAATAAT ACTATTGTTG ATATCCAGAA TTCTGCATAC
ACCTCACAAA ATGACAACTG TTTTTTCTTC TTTTAATGTT
CTCTTGATTT GTTGTTTGGT TTTTAATTAT AAGATATAAT
TGATTAATTC TATTTATTAA AAAAGTTAAT TATTAGTATT
ATGTTGGTTT ATAAATATAG TATTTTTTAA ATTTATTCTT
AACTTTATTG AAAGTCTAAC AATGTAAGTT TTTTAATTTT
CATAAGAGAA AAAGAAAATC AAGAAATTTT AATTAAAAAT
AATTAATGTA TATGAAAAAT GAAATAAAGT CTTATAAGAT
GTTGAAACAG AGAGTGTTTG GATAGAAATT AAAGAAAGGG
CTAGGCTATA CTATAGGGAA AGAGAAATAG GGAAGTGTGA
AGTGAGGGGG CGCCGTCAAC CAATGAGAGT GCCATAAACA
CAACAAAAAC CCGTTTCCCG TTTCTCACTT ATTCATGGGA
CCCACATAAC CGTACGGAGC GTCTCCCAAC GTTACGCCCT
GTCAACACGA GACATCAACA CTCTCATACT TGCTCAATTC
TAGCTCGACA ACGCATTGTA CCTTAACCCT TCTACTAATC
ACAACTCGAC AACGCATCGT ACCTTAAATT CTCATTCCTT
TCCCCAATTT TTATTCTTAT TCTTCTTTTT TCCTTCACAA
TTCAAAAATA GAAAAAGGAA ATTCCCATGC TATCCTATTA
TTAGACACCC TCCACTTCTT TTGCTTTCGC GTTCGTTTTC
TCTTTCTCGG TCTTGCTTTG CTCAGGTGAA TACCACTCTC
TCACTCTACT ACTTCCTCTC TAGCTAGGGT TTCCTTCTTT
ATACAAAACA CAACCTAACA AGTAACAACC TTCTTTATAT
GTACTTATTC CTTAACCCTC CTGTTACCCT TTTTAGCTAT
TTCTATTTGT GCTGCTTTCA TAGAATTGCT AGTTACGTGG
GACAATTAAG CACTAAGAAG
SEQ ID NO: 2 Sequence of Dt2^{HapII} promoter
CCACTCCAAC AGGTATAAAA AGAAAGTTAA GCCAAAGAGA
AAACACACCA CCCTGGAGAC TCAGAGCTCT CTTATGAATA
CATCCTGTCT GGGTGTCTCT AGTAGGGGAA ATCTTTCTTT
TTCCATCCCT TCTCTTCCAT TAGTTTCTAA ACCCCTTTTC
AAGTGTAAGG CTCCTTATGG CTATGAGAGA CTAAACCCTT
AGTTAGGGTC TGACAGGTCT AAAAAGCCAA AAGATGTATT
GCACACTTCA TATCTATCAA TGCAAACAAG TGTTTTCTTT
CCTATTATCC TTTCTTATTT TAATTTCATG TATCATTCAT
CCTTGCACCA TCTTCAGGGG TTAGGTGTTT GATAGAGGGT
AATCCTTAAT AAAAATACAA GAAAGCTCTT ACATGCATCA
GTTTTAAGGA TTAGTCGCTC GACAGAGGAT AATTTCTAAT
AGAACTAAAG GGAAGATGTA TCTTAATAAA ATCATTGCTA
GACATAGAGT GATTGCATTA TGTCTATGCA TCAAAGCAAA
CATCTAGAAT TAAAACTTCA TGCATCTTAT CTATCGGGTC
TTTGCAAAGA CATTTGGGAA ATAGATAGAT AGGTAAGTTA
GGTTTGTCAT CGTGGGATAT CATGGGCAAA GTATTCTAAT
AGATGTGGGT AGGAAAAAAA ATCACTAAAT TGATAATGAA
AAAAATCTAA AATAATACAT CTTAGACAAA TAAAGCAGGC
TAAGTCCCGA CATTATCACA TCTTGAATTT ATCTTTCTTT
ATCTTCATCT TCATTTTTTA TTTTTCTTAT CTTTTACTTT
TCTTATCTTA TCTTTTTTTA TCTTTATCAT CTTTTAATTT
AAATATTTTA TCTTTTATAT CTTATCTTTT CTTTTAAATT
TTTATTTTAT CTTTTATCTT TCTGCATCTT TTATTTTAAA
ATTCTTATCT ATTGCTTTTA ATTTGGGTTT GTATCAATCT
AAATCCAAAC AAATTCTGGG TGGATTCAAC ACTCAAACTT
TCAAATACTT TACTACTTAT GGTAAATTTG ATGCACTTGA
TAACGAGTTA ACATTTGTCA CCATATCAAC TAGTCATATA
TGAAAAATTT GTGCAATTAT ATATATATAT ATATATATAT
ATATATATAT ATAATATTTT TAGTTTCTTA TATATTTATA
AACTATTAAG TCGTGAAAAA ATAAGGAACT AAAAATATAA
ATTACAAAAA TTATAGACAT AAGTAAATGA TGTGTGAGTT
CTTGTGTTTG TCAATGTAAT TATCATATCA TCAATTAGGG
GTAATATAAT TAATAATAGA GACCAAAAAT TTGTTTAGGG
ATTTAAAACT ATACATGTTC AAACAAGATT ATGAAGACTA
AAAAACATTT TTTTTTAAAA AAAGATTACA CATAATTATT
CTATTAGATA TTTGATAATA ACAAATTAAT GAATTGTCAA
TATATTGTGT GATAGTTAAC ACTCTTAATT AACAAAAGGG
ACTAAAAGTG GTAACTAATA AAAATTTAAT TTAGATTAGG
ACTAAAAGCA AAAATTTTAA AATTTTAAGA AACAAAAAGA
TAATAAATTC AAACAAATAA ATGCATGTTT AATTTGAAAT
TGAAATTATT ATATTCTGTG TACTAATGCA GATCCATCGT
ATAAAAAAAT GAAAACAAAA ATAATGGTTA TATATAATCC
TTTTGCAAAA GTATTTTTAC CTCAAAGCTA TTTTTATTTG
ATTTCAAACG TGATTTAACA CACATGCAAT ATGCAATACA
CAACTTATTT TTCCAAATAT TTTAAAAAAT AATAACACAA
CTCAGGGGTT TTGAACATAA AGATTCAATT GACATGTGAA
AAATGGACCT AGTCCCTTCC ATAGCATCTG ATTGACATGT
GAAAAATGGA CCTAATCCCT TTCCATAGCA TCTGATTGAC
ATAAGCCAAA CATGAATAGG AAATTAGACG AATTTAGTGA
CGGAGCCACA AGCTAGCAAG AGGCCAAACA AAGTAACAAA
AACAATATTA ACTTCATTTT TATAAATCAA GTTATTTATT
ATTCGAAACA AATCCCGAAA ATCCAAACTA CTAATTATCC
AAGACCTAAA ATTCCCAATT ATAAACTAAA ATTCCCTTCT
AGTTACAAAT GCTTTTTAAC AAGCATTTTC CCGAATAGGA
CGTGTGAACC AACAAAACAC ACGTTAATGT AATATTAACA
TATTATTTTT TAAAAAAATA ATAACACATT CACACAAATA
CATATACACA AAAAGTGAAA AATAAACTCC AAATGAAATA
AAAATTAATA GGGTTTATCA TCTTTTTGTT TCAATTTTTT
ATAAAAAAGA AAAATTGTTT TTAGTCTCTT TAAAAAAATT
GTTCCATTTT TGCTCCTATT TTGTGAGAGT AAAAACGGAA
TGAAAGTGTT TTTGAGAGAA AAAAATGAAA CAGTTTTTTC
AAGAACTAAA ATTAGTCACT AAAAAATTGA CAAGAGATTA
AAAACAGAAA TTCACGATGA AGACAAAAGA GTTAATAACC
CTAAAATAAT ACTATTGTTG ATATCCAGAA TTCTGCATAC
ACCTCACAAA ATGACAACTG TTTTTTCTTC TTTTAATGTT
CTCTTGATTT GTTGTTTGGT TTTTAATTAT AAGATATAAT
TGATTAATTC TATTTATTAA AAAAGTTAAT TATTAGTATT
ATGTTGGTTT ATAAATATAG TATTTTTTAA ATTTATTCTT
AACTTTATTG AAAGTCTAAC AATGTAAGTT TTTTAATTTT
CATAAGAGAA AAAGAAAATC AAGAAATTTT AATTAAAAAT
AATTAATGTA TATGAAAAAT GAAATAAAGT CTTATAAGAT
GTTGAAACAG AGAGTGTTTG GATAGAAATT AAAGAAAGGG
CTAGGCTATA CTATAGGGAA AGAGAAATAG GGAAGTGTGA
AGTGAGGGGG CGCCGTCAAC CAATGAGAGT GCCATAAACA
CAACAAAAAC CCGTTTCCCG TTTCTCACTT ATTCATGGGA
CCCACATAAC CGTACGGAGC GTCTCCCAAC GTTACGCCCT
GTCAACACGA GACATCAACA CTCTCATACT TGCTCAATTC
TAGCTCGACA ACGCATTGTA CCTTAACCCT TCTACTAATC
ACAACTCGAC AACGCATCGT ACCTTAAATT CTCATTCCTT
TCCCCAATTT TTATTCTTAT TCTTCTTTTT TCCTTCACAA
TTCAAAAATA GAAAAAGGAA ATTCCCATGC TATCCTATTA
TTAGACACCC TCCACTTCTT TTGCTTTCGC GTTCGTTTTC
TCTTTCTCGG TCTTGCTTTG CTCAGGTGAA TACCACTCTC
TCACTCTACT ACTTCCTCTC TAGCTAGGGT TTCCTTCTTT
ATACAAAACA CAACCTAACA AGTAACAACC TTCTTTATAT
GTACTTATTC CTTAACCCTC CTGTTACCCT TTTTAGCTAT
TTCTATTTGT GCTGCTTTCA TAGAATTGCT AGTTACGTGG
GACAATTAAG CACTAAGAAG
SEQ ID NO: 3 Dt2^{HapI-1}-CDS
ATGGGAAGGG GTAGGGTTCA GCTGAAGCGG ATCGAGAACA
AAACAAGCCA GCAAGTGACG TTTTTCAAGC GTAGATCGGG
ACTTCTCAAG AAAGCCAGCG AAATCTCTGT GCTATGTGAT
GCTCAAGTTG CTTTGATTAT ATTTTCTACC AAAGGCAAAC
TTTTTGAGTA TTCCTCTGAA CGCAGCATGG AAGACCTCCT
TGAACGTTAC GAGAGATGTA GCCATACAGC ACTTGCTGGA
GCTAACAATG TCGAATCACC GGGATTTTGG TCTTTCGAAC
ATATCAAGCT CACCGCTAAA GTTGAAGTCT TGGAGAGGAA
CATAATGAAT TTCTTTGGAA ATGATCTGGA TCCCTTGAGT
TTGAAAGAGC TTCACAGTTT GGAGCAGCAG ATTGAGACAT
CTCTGAAGCG CATCCGAACT AGAAAGAATC AAGTTATGAA
TCAATCCGTC TCAGACCTGC ATAAAAAGGC AAGGACATTA
CAAGTGCAAA ACAGATGGCT AGGAAAGATG AAGGAGAAAG
CGAAGACAGT GACTGAAGGT CCACACAACG GCCCAGAAAC
TCTAGGCTTT GATTCATCCA CACTCAACTT ATCTTCTCCA
CAGCTACCAC CACCACCATC ACCACAAAGA CTGGTTCCTT
CTCTAACTCT CAGTGAGACA ATGCAAGGAG GAACACCAGT
GGAAGAAACG GGTGAGGCTC AAACAGTCCC TAGTGGCAAT
TCTCTCATCC CACCATGGAT GCTGCGCTGC ATGTCTGACT AG
SEQ ID NO: 4 Dt2^{HapI-2}-CDS
ATGGGAAGGG GTAGGGTTCA GCTGAAGCGG ATCGAGAACA
AAACAAGCCA GCAAGTGACG TTTTTCAAGC GTAGATCGGG
ACTTCTCAAG AAAGCCAACG AAATCTCTGT GCTATGTGAT
GCTCAAGTTG CTTTGATTAT ATTTTCTACC AAAGGCAAAC
TTTTTGAGTA TTCCTCTGAA CGCAGCATGG AAGACCTCCT
TGAACGTTAC GAGAGATGTA GCCATACAGC ACTTGCTGGA
GCTAACAATG TCGAATCACC GGGATTTTGG TCTTTCGAAC
ATATCAAGCT CACCGCTAAA GTTGAAGTCT TGGAGAGGAA
CATAATGAAT TTCTTTGGAA ATGATCTGGA TCCCTTGAGT
TTGAAAGAGC TTCACAGTTT GGAGCAGCAG ATTGAGACAT
CTCTGAAGCG CATCCGAACT AGAAAGAATC AAGTTATGAA
TCAATCCGTC TCAGACCTGC ATAAAAAGGC AAGGACATTA
CAAGTGCAAA ACAGATGGCT AGGAAAGATG AAGGAGAAAG
CGAAGACAGT GACTGAAGGT CCACACAACG GCCCAGAAAC
TCTAGGCTTT GATTCATCCA CACTCAACTT ATCTTCTCCA
CAGCTACCAC CACCACCATC ACCACAAAGA CTGGTTCCTT
CTCTAACTCT CAGTGAGACA ATGCAAGGAG GAACACCAGT
GGAAGAAACG GGTGAGGCTC AAACAGTCCC TAGTGGCAAT
TCTCTCATCC CACCATGGAT GCTGCGCTGC ATGTCTGACT AG
SEQ ID NO: 5 Sequence of Dt2^{HapI-1}-protein
MGRGRVQLKR IENKTSQQVT FFKRRSGLLK KANEISVLCD
AQVALIIFST KGKLFEYSSE RSMEDLLERY ERCSHTALAG
ANNVESPGFW SFEHIKLTAK VEVLERNIMN FFGNDLDPLS
LKELHSLEQQ IETSLKRIRT RKNQVMNQSV SDLHKKARTL
QVQNRWLGKM KEKAKTVTEG PHNGPETLGF DSSTLNLSSP
QLPPPPSPQR LVPSLTLSET MQGGTPVEET GEAQTVPSGN
SLIPPWMLRC MSD
SEQ ID NO: 6 Sequence of Dt2^{HapI-2}-protein
MGRGRVQLKR IENKTSQQVT FFKRRSGLLK KASEISVLCD
AQVALIIFST KGKLFEYSSE RSMEDLLERY ERCSHTALAG
ANNVESPGFW SFEHIKLTAK VEVLERNIMN FFGNDLDPLS
LKELHSLEQQ IETSLKRIRT RKNQVMNQSV SDLHKKARTL
QVQNRWLGKM KEKAKTVTEG PHNGPETLGF DSSTLNLSSP
QLPPPPSPQR LVPSLTLSET MQGGTPVEET GEAQTVPSGN
SLIPPWMLRC MSD
SEQ ID NO: 7 Sequence of primer F1
ATGGGAAGGGGTAGGGTTC
SEQ ID NO: 8 Sequence of primer R1
CTAGTCAGACATGCAGCGCA
SEQ ID NO: 9 Sequence of target primer sgRNA1
tgtagccatacagcacttgc
SEQ ID NO: 10 Sequence of target primer sgRNA2
aggaacaccagtggaagaaa
SEQ ID NO: 11 PMDC123-HindIII-gRNA-F
TGTAAAACGACGGCCAGTGCCAAGCTTACGACTCACTATAGGGCG
AATTG
SEQ ID NO: 12 PMDC123-pstI-gRNA-R
GTGCTCCACCATGTTGACCTGCAGAACAAAAGCTGGAGCTCACTA
GT
SEQ ID NO: 13 Sequence of DN50 in FIG. 5C
TGTAGCCATACAGCACTTGCTGGAGCTAA
SEQ ID NO: 14 Sequence of Dt2_{CR-1} in FIG. 5C
TGTAGCCATACAGCACTGCTGGAGCTAA
SEQ ID NO: 15 Sequence of DN50 in FIG. 5D
TGTAGCCATACAGCACTTGCTGGAGCTAACAATGTCGAAT
SEQ ID NO: 16 Sequence of Dt2^{CR-2} in FIG. 5D
TGTAGCCATACAATGTCGAAT

### REFERENCES

Becker A, Theißen G. (2003). The major clades of MADS-box genes and their role in the development and evolution of flowering plants. Mol Phylogenet Evol. 29(3):464-89.
Banfield, M. J., Barker, J. J., Perry, A. C., and Brady, R. L. (1998). Function from structure the crystal structure of human phosphatidylethanolamine-binding protein suggests a role in membrane signal transduction. Structure 6, 1245-1254.
Liu, Y., Zhang, D., Ping, J., Li, S., Chen, Z., and Ma, J. (2016). Innovation of a regulatory mechanism modulating semi-determinate stem growth through artificial selection in soybean. PLOS genetics 12, e1005818.
Ma, X., Zhang, Q., Zhu, Q., Liu, W., Chen, Y., Qiu, R., Wang, B., Yang, Z., Li, H., Lin, Y., et al. (2015). A Robust CRISPR/Cas9 System for Convenient, High-Efficiency Multiplex Genome Editing in Monocot and Dicot Plants. Mol Plant 8, 1274-1284.
Ping, J., Liu, Y., Sun, L., Zhao, M., Li, Y., She, M., Sui, Y., Lin, F., Liu, X., Tang, Z., et al. (2014). Dt2 is a gain-of-function MADS-domain factor gene that specifies semideterminacy in soybean. The Plant cell 26, 2831-2842.
Zeng, X., Liu, H., Du, H., Wang, S., Yang, W., Chi, Y., Wang, J., Huang, F., and Yu, D. (2018). Soybean MADS-box gene GmAGL1 promotes flowering via the photoperiod pathway. BMC genomics 19, 51.
Zhang, D., Wang, X., Li, S., Wang, C., Gosney, M. J., Mickelbart, M. V., and Ma, J. (2019). A Post-domestication Mutation, Dt2, Triggers Systemic Modification of Divergent and Convergent Pathways Modulating Multiple Agronomic Traits in Soybean. Mol Plant 12, 1366-1382.
Zheng Q, Zheng Y, Perry SE. (2013). Decreased GmAGL15 expression and reduced ethylene synthesis may contribute to reduced somatic embryogenesis in a poorly embryogenic cultivar of Glycine max. Plant Signal Behav. 8(9):e25422.
Jie Gao (2017). Effects of spring and summer sowing on the growth period of different soybean germplasms and preliminary mapping of soybean petiole length QTL. Master's thesis. Liaocheng University.
Pengtao Gong (2005). Genetic control of branch development in plants[J]. Molecular Plant Breeding.

1. An isolated gene for regulating branch numbers of legume, wherein a coding region sequence of the gene is shown in SEQ ID NO:3 or SEQ ID NO:4 or homologous sequences thereof.

2. An isolated protein that regulates branch numbers of legume, wherein an amino acid sequence of the protein is shown in SEQ ID NO:5 or SEQ ID NO:6 or homologous sequences thereof.

3. An isolated promoter of a gene that regulates branch numbers of legume, wherein a sequence of the promoter is shown in SEQ ID NO: 1 or SEQ ID NO: 2.

4. An expression vector, wherein the expression vector comprises a sgRNA target sequence designed for the gene according to claim 1.

5. The expression vector according to claim 4, wherein the expression vector has an antibiotic marker or a chemical resistance marker.

6. Use of the gene for regulating branch numbers of legume according to claim 1, an isolated protein that regulates branch numbers of legume, wherein an amino acid sequence of the protein is shown in SEQ ID NO:5 or SEQ ID NO:6 or homologous sequences thereof, or an isolated promoter of a gene that regulates branch numbers of legume wherein a sequence of the promoter is shown in SEQ ID NO: 1 or SEQ ID NO: 2, in regulating branch numbers of legume or improving yield of legume.

7. A method for improving yield of legume, or regulating branch numbers of legume, wherein the method comprises a step of inactivating the function of the gene according to claim 1.

8. A method for improving yield of legume, or regulating branch numbers of legume, wherein the method comprises:
a. designing an sgRNA target sequence for gene according to claim 1, and introducing the sgRNA target sequence into an expression vector;
b. introducing the expression vector into Agrobacterium tumefaciens; and
c. transfecting legume, with the Agrobacterium tumefaciens.

9. The method according to claim 8, wherein number of the sgRNA target sequence is more than one.

10. The method according to claim 8, wherein the sgRNA target sequence is as shown in SEQ ID NO:9 or SEQ ID NO:10.

11. The use of claim 6, wherein the legume is a plant of genus Glycine, Pisum, or Cicer Linn.

12. The use of claim 6, wherein the legume is soybean, pea, mung bean, broad bean, black bean, or chickpea.

13. The method of claim 7, wherein the legume is a plant of genus Glycine, Pisum, or Cicer Linn.

14. The method of claim 7, wherein the legume is soybean, pea, mung bean, broad bean, black bean, or chickpea.

15. The method of claim 7, wherein the function of the gene is inactivated by gene knockout.

16. The method of claim 8, wherein the legume is a plant of genus Glycine, Pisum, or Cicer Linn.

17. The method of claim 8, wherein the legume is soybean, pea, mung bean, broad bean, black bean, or chickpea.
